(19) United States
(12) Patent Application Publication
(10) Pub. No.: US 2024/0133818 A1
(43) Pub. Date: Apr. 25, 2024

(54) ACTIVATED-QUENCHED POLYSACCHARIDE AND IMPROVED METHODS FOR QUANTIFICATION OF POLYSACCHARIDE IN A VACCINE COMPOSITION

(71) Applicant: Biological E Limited, Hyderabad, Telangana (IN)

(72) Inventors: Rajendar BURKI, Hyderabad, Telangana (IN); Jaya Sheela PYDIGUMMALA, Hyderabad, Telangana (IN); Sreenivasa Rao GANTI; Siva Prasad KANNURI, Hyderabad, Telangana (IN); Shravan Kumar RENUKUNTLA, Hyderabad, Telangana (IN); Ramesh Venkat MATUR, Hyderabad, Telangana (IN); Narender Dev MANTENA, Hyderabad, Telangana (IN); Mahima DATLA, Hyderabad, Telangana (IN)

(21) Appl. No.: 18/277,834

(22) PCT Filed: Feb. 25, 2022
(86) PCT No.: PCT/IN2022/050168

§ 371 Date: Aug. 17, 2023

(30) Foreign Application Priority Data
Feb. 26, 2021 (IN) 202141008155

(51) Int. Cl. G01N 21/82; G01N 33/15

(52) U.S. Cl. CPC G01N 21/82; G01N 33/15

(57) ABSTRACT

The present invention provides a novel reference standard, comprising of activated-quenched polysaccharide, for quantifying polysaccharide content in a vaccine composition using nephelometry. The invention also provides a method for preparing the activated-quenched polysaccharide, for use as a reference standard. Further, a nephelometry based method for quantifying the polysaccharides in a multivalent conjugate vaccine is also provided. The reference standard of the present invention, comprising of the activated-quenched polysaccharide, is stable and can be used for accurate quantification of polysaccharides through nephelometry.

### FIELD OF THE INVENTION

The present invention relates to a reference standard, comprising of activated-quenched polysaccharide, and improved method for quantitative estimation of individual polysaccharides in a vaccine, using nephelometry.

### BACKGROUND OF THE INVENTION

Nephelometry is a technique that is used for quantifying an analyte based on the principle of light scattering by the particles in the solution. A dilute suspension of small particles will scatter light passed through it rather than simply absorbing it. The amount of scatter is determined by collecting the light at an angle. The concentration of the analyte in a sample is proportional to the intensity of the scattered light, which is measured by a detector. (Whicher et. al., Crit Rev Clin Lab Sci. 1983; 18(3):213-60).

Nephelometry has been applied for the quantitative determination of various proteins and other antigens in blood serum, urine or cerebrospinal fluid such as lipoproteins, immunoglobulins, complement factors, rheumatoid factors and immune complexes. This technique is widely used in clinical laboratories because it is relatively easily automated. (Whicher et. al., Ann Clin Biochem. 1980 July; 17(4):170-7.) (Vergani et. al., J Clin Pathol. 1983 July; 36(7): 793-797.)

In immunonephelometry, antibodies and the antigen are mixed in concentrations such that only small aggregates are formed that do not quickly settle to the bottom. The amount of light scatter is measured and compared to the amount of scatter from known reference standards. The amount of the unknown is determined from a standard curve. Nephelometry, is best performed in a dilute suspension of particles where self-absorption and reflection are minimal. Under these conditions, the relationship between the concentration of scattering particles and scattered light intensity is almost linear over a very wide range.

Endpoint (or fixed-time) nephelometry measures the maximum scattered light after an antigen-antibody reaction has reached equilibrium, or after a fixed reaction time. The antigen concentration in the unknown sample is calculated from a response curve produced with reference standards containing known amounts of the antigen tested under identical conditions.

Rate or kinetic nephelometry is an alternative method in which the peak rate of immune-complex formation is measured. The peak rate of the immune complex formation is proportional to the concentration of antigen.

Lee et. al., (J Biol Stand. 1983 January; 11(1):55-64) discloses a rate nephelometry-based method for the measurement of the individual pneumococcal, as well as meningococcal, polysaccharides in the polyvalent vaccine. The moisture-corrected weights of the polysaccharides were used in preparing reference standard. While preparing the standard curves for quantitative assessment, the optimum concentration for pneumococcal polysaccharides of serotypes 1, 2, 3, 4, 6A, 7F, 8, 9N, 12F, 14, 18C, 19F, 23F and 25 were found to be 1-5 ug/mL. The optimum concentration for meningococcal polysaccharides, groups A, C, Y and W 135, was found to be 1-4 ug/mL.

Salerno et al., (J Biol Stand. 1984 October; 12(4):447-50) describes an automated rate nephelometric method for quantitative analysis of the 23-valent pneumococcal vaccine (Pneumovax). A polyvalent standard curve was prepared by diluting individual polysaccharide types in distilled water to 1 mg/ml concentration corrected for moisture; 1 ml of each polysaccharide was pooled and diluted with Beckman ICS diluent to yield a final concentration of 10 ug/ml of each polysaccharide.

Lee et. al., (Biologicals. 2002 Jim; 30(2):97-103) discloses a rate nephelometry-based method for quantitative analysis of individual polysaccharides in a polyvalent pneumococcal conjugate vaccine (Prevnar) using monovalent conjugate without aluminium phosphate adjuvant as standard. The concentration of individual serotype polysaccharides 4, 6B, 9V, 14, 18C, 19F and 23F in the conjugate vaccine was determined to be between 82.3 to 119% of the manufacturer's indicated values. The 7-valent pneumococcal conjugate vaccine was treated with trypsin and kept at 25° C. for 17 h to digest and release polysaccharide-peptide fragments into solution. This was subsequently centrifuged at 3000 rpm for 5 minutes to remove the aluminium phosphate adjuvant. The concentration of individual polysaccharide in the sample was then determined by rate nephelometry.

Tabatabaie et al. (Iran J Allergy Asthma Immunol. 2008 June; 7(2): 69-77) discloses a study to investigate humoral immune function in bronchiectatic patients who have been administered unconjugated pneumococcus polyvalent vaccine (PNEUMO 23® Aventis, Pasteur, France). Immunoglobulin isotypes concentration in blood samples of 40 patients was measured by nephelometry.

Chen et. al., (Chinese Journal of Biologicals. 2103 26(8):1170-1174) discloses the effect of various desorption treatments while quantifying the polysaccharides in pneumococcal conjugate vaccine. Heptavalent pneumococcal conjugate vaccine, comprising of serotypes 4, 6B, 9V, 14, 18C, 19F and 23F, was desorbed using trypsin or sodium hydroxide and the results were compared. Pneumococcal serotype polysaccharides were used to plot standard curve. Results showed that the polysaccharide contents of the untreated samples were lower than the treated ones except for serotype 14. The polysaccharide contents of trypsin treated samples were lower than the sodium hydroxide treated ones except for serotype 23F.

Chen et. al., (Chinese Journal of Biologicals. 2015 28(7):718-722) discloses a nephelometry based method for quantifying serotype polysaccharide content in 13-valent pneumococcal conjugate vaccine. Serotype polysaccharide mix, comprising of serotypes 1, 3, 4, 5, 6A, 6B, 7F, 9V, 14, 18C, 19A, 19F and 23F, along with aluminium phosphate adjuvant was used as reference standard. Sodium hydroxide based desorption method was used. The recovery of all the 13 serotype polysaccharide was found to be between the allowed range of 70%˜130%.

Antony et. al., (Clin Biochem. 2017 January; 50(1-2):80-83) discloses purification and characterisation of anti-pneumococcal capsular polysaccharide IgG immunoglobulins. It provides a method for determining the anti-pneumococcal antibody immunoglobulin composition, using nephelometry, in patients immunized with Pneumovax.

Fortpied et al. (Hum Vaccin Immunother. 2018; 14(5):1243-1250) discloses the stability of Protein-D-conjugated pneumococcal vaccine (Synflorix, GSK) exposed to subzero temperatures. Rate nephelometry was used to assess the antigen concentration and antigenicity of the conjugate vaccine after exposure to subzero temperatures. The vaccine sample was prepared for analysis by incubating the sample with a protease (trypsin or pronase E depending on the serotype) and subsequently centrifuging it to remove the aluminium-salt particles. Standard curve was generated using vaccine samples with known concentrations of antigens.

Chinese Patent Application No. 106018832A discloses a method for detecting the quantity of various types of serotype polysaccharide in polyvalent pneumococcal conjugate vaccine. The method uses polysaccharide-protein conjugates for preparing the reference standard. In this method, a solution having a known concentration of monovalent conjugate is prepared. A serially increasing volume of the standard solution of serotype, which is to be quantified, is added to a predetermined volume of conjugate vaccine, to plot a standard curve and determine the quantity of polysaccharide in vaccine composition.

U.S. Pat. No. 8,562,999 discloses a rate nephelometry for determining the antigenicity of the polysaccharide conjugates in a 13 valent pneumococcal conjugate vaccine, comprising of polysaccharides from serotypes 1, 3, 4, 5, 6A, 6B, 7F, 9V, 14, 18C, 19A, 19F and 23F, adsorbed onto aluminium phosphate adjuvant. In order to prepare the sample for rate nephelometry analysis, adjuvant is solubilised by addition of 1M NaOH and then immediately neutralised with 1M citric acid.

European patent No. 0497525B1 uses nephelometry for quantifying the polysaccharide content in pneumococcal conjugate vaccine. It uses monovalent conjugate of respective polysaccharide as a standard. The alum-adsorbed samples are solubilised by dialysis against 3% sodium citrate for 6h in order to prepare the sample for nephelometric analysis.

As discussed above, nephelometry is widely used for various applications in the process of quality control of vaccines. The prior art documents mostly mention the use of monovalent conjugates as a reference standard for quantifying polysaccharides in a conjugate vaccine. However, the carrier protein in the monovalent conjugate reference standard, as used in the prior art, is prone to degradation over time. This results in the faulty estimation of polysaccharides in the conjugate vaccine composition, which is undesirable. Therefore, there is a need for standard references, which remain stable, for accurate quantification of polysaccharides in a vaccine composition, using nephelometry.

### OBJECTIVE OF THE INVENTION

It is the main objective of the present invention to provide for a stable reference standard for quantification of polysaccharides in a vaccine composition, using nephelometry.

Another objective of the invention is to provide a method for preparing the stable reference standard for quantifying polysaccharides in a vaccine composition, using nephelometry.

Yet another objective of the present invention is to provide a nephelometry based method for quantifying the polysaccharides in a vaccine composition, using the novel reference standard of the present invention.

### SUMMARY OF THE INVENTION

The present invention provides a reference standard for quantification of polysaccharides in a vaccine composition using nephelometry, which comprises an activated-quenched polysaccharide.

In an embodiment, the invention provides a reference standard for quantification of polysaccharides in a vaccine composition, which comprises an activated-quenched polysaccharide obtained by quenching an activated polysaccharide with a quenching agent wherein the quenching agent is an amino acid.

In an embodiment, the invention provides a reference standard for quantification of polysaccharides in a vaccine composition, wherein the activated-quenched polysaccharide is prepared by a method, comprising the steps of:
a) treating a polysaccharide with an activating agent to obtain an activated polysaccharide; and
b) quenching the activated polysaccharide from step (a) with a quenching agent to obtain the activated-quenched polysaccharide.

In an embodiment, the present invention provides a method for preparing a reference standard comprising of an activated-quenched polysaccharide, for quantifying polysaccharide in a vaccine composition, wherein the said method comprises the steps of:
a) treating a polysaccharide with an activating agent to obtain an activated polysaccharide; and
b) quenching the activated polysaccharide from step (a) with a quenching agent to obtain the activated-quenched polysaccharide.

In an embodiment, the invention provides a nephelometry-based method for quantifying polysaccharides in a vaccine composition, using the activated-quenched polysaccharide as a reference standard.

In an embodiment of the present invention, the activating agent is selected from a cyanylating agent, oxidising agent, reducing agent and condensing reagents.

In an embodiment of the present invention, the cyanylatingagent is 1-cyano-4-dimethylamino-pyridinium tetrafluoroborate (CDAP), cyanogen bromide, N-cyano trimethyl ammonium tetrafluoroborate (CTEA) and p-nitro phenyl cyanate (pNPC), preferably CDAP.

In an embodiment of the present invention, the quenching agent is an amino acid.

In an embodiment, the invention provides a method for quantifying the polysaccharide content in a vaccine composition, the said method comprising the steps of:
a. reacting the polysaccharide specific sera with various pre-determined concentrations of the reference standard of the present invention, and determining the light-scattering rate in a nephelometer to obtain a standard curve;
b. reacting the vaccine drug product with polysaccharide specific sera and determining the light-scattering rate in a nephelometer; and
c. comparing the light-scattering rate obtained from step b. with the standard curve obtained from step a. to obtain the polysaccharide content in the conjugate vaccine composition.

### BRIEF DESCRIPTION OF THE DRAWINGS

FIG. 1 depicts comparative data on quantification of individual serotype polysaccharide in conjugate vaccine drug product using three different reference standards. Native polysaccharide, conjugate polysaccharide and activated-quenched polysaccharide were used as reference standard.

FIG. 2 depicts comparative data on quantification of individual serotype polysaccharides in conjugate vaccine drug product using 0 day and 3 months old standards. Conjugate polysaccharide and activated quenched polysaccharide was used as the reference standard.

FIG. 3 depicts the process for preparation of activated-quenched polysaccharide.

FIG. 4 depicts the scheme for determination of polysaccharide content in conjugate vaccine.

FIG. 5 depicts the schematic illustration of activated quenched polysaccharide preparation.

### DETAILED DESCRIPTION OF THE INVENTION

The present invention relates to a stable reference standard for quantification of polysaccharides in a vaccine composition, using nephelometry.

More specifically, the invention relates to an activated-quenched polysaccharide as a stable reference standard, which is used for the quantification of polysaccharides in a vaccine composition.

In yet another embodiment, the present invention also provides a nephelometry-based method for quantifying polysaccharides in a vaccine composition using the activated-quenched polysaccharide as the reference standard.

The activated-quenched polysaccharide reference standard of the instant invention is more stable compared to polysaccharide conjugate-based reference standards disclosed in the prior art. Also, replacing bulky carrier protein, with amino acids such as glycine, in the reference standard, reduces interference and provides the polysaccharide specific antibodies a better access to the epitopes. This helps in more accurate quantification of the polysaccharide content in the conjugated/unconjugated vaccine composition.

In an embodiment, the reference standard of the present invention is stable for a period of at least three months.

In an embodiment, the source of polysaccharide is from a bacterial polysaccharide selected from, but not limited to, Neisseria meningitidis, Streptococcus pneumoniae, Haemophilus influenzae type b, and Salmonella typhi, yeast, filamentous fungus, algae or plant cells. In yet another embodiment, the polysaccharide is pneumococcal polysaccharide from one or more pneumococcal serotypes selected from 1, 2, 3, 4, 5, 6A, 6B, 6C, 7F, 8, 9N, 9V, 10A, 11A, 12F, 14, 15A, 15B, 15C, 16F, 17F, 18C, 19F, 19A, 20A, 20B, 22F, 23A, 23B, 23F, 24B, 24F, 31, 33F, 34, 35B, 35F, 38, 39 and 45 and the like.

In yet another embodiment, the polysaccharide from Salmonella is selected from Vi, 02 and the like.

In yet another embodiment, the polysaccharide from Neisseria meningitidis is selected from serotypes A, C, W, X, Y and the like.

In a more preferred embodiment, the present invention relates to activated-quenched polysaccharide reference standard for nephelometry, which is prepared from pneumococcal capsular polysaccharides.

In an embodiment, the polysaccharide is conjugated to a carrier protein to prepare the conjugate vaccine.

In some embodiments, the carrier protein is selected from one or more of the following carrier proteins, PsaA, CRM₁₉₇, inactivated bacterial toxins such as tetanus toxoid, pertussis toxoid; cholera toxoid, exotoxin A from Pseudomonas aeruginosa, bacterial outer membrane proteins such as outer membrane complex C (OMPC), porins, transferrin binding proteins, pneumolysin, PspA, C5a peptidase from Group A or Group B streptococcus, or Haemophilus influenzae protein D, ovalbumin, keyhole limpet hemocyanin, (KLH), bovine serum albumin (BSA), purified protein derivative of tuberculin (PPD) and the like.

The activated-quenched polysaccharide reference standard of the instant invention can be used for quantification of polysaccharides in conjugate vaccine composition prepared by conjugating any of the above said polysaccharides with any of the above said carrier proteins.

In an embodiment, the invention provides a reference standard, comprising of an activated-quenched polysaccharide prepared by a method, comprising the steps of:
a) treating a polysaccharide with an activating agent to obtain an activated polysaccharide; and
b) quenching the activated polysaccharide from step (a) with a quenching agent to obtain the activated-quenched polysaccharide.

In an embodiment, the invention provides a reference standard, comprising of an activated-quenched polysaccharide prepared by a method, comprising the steps of:
a) treating a polysaccharide with an activating agent to obtain an activated polysaccharide; and
b) quenching the activated polysaccharide from step (a) with an amino acid to obtain the activated-quenched polysaccharide.

In an embodiment of the present invention, the activating agent is selected from a cyanylating agent, oxidising agent, reducing agent and condensing reagents.

In an embodiment of the present invention, the cyanylatingagent is selected from the group comprising 1-cyano-4-dimethylamino-pyridinium tetrafluoroborate (CDAP), cyanogen bromide, N-cyano trimethyl ammonium tetrafluoroborate (CTEA) and p-nitro phenyl cyanate (pNPC), preferably CDAP.

In an embodiment of the present invention, the quenching agent is a an amino acid selected from a group comprising glycine, lysine, alanine and the like.

In an embodiment, the present invention provides a method for preparing the activated-quenched polysaccharide, wherein the said method comprises the steps of:
a) treating a polysaccharide with an activating agent to obtain an activated polysaccharide; and
b) quenching the activated polysaccharide from step (a) with an amino acid to obtain the activated-quenched polysaccharide.

In an embodiment, the present invention provides a method for preparing the reference standard, comprising of an activated-quenched polysaccharide, wherein the said method comprises the steps of:
a) treating a polysaccharide with 1-cyano-4-dimethylamino-pyridinium tetrafluoroborate (CDAP) to obtain an activated polysaccharide; and
b) quenching the activated polysaccharide from step (a) with an amino acid selected from a group comprising glycine, lysine, alanine and the like to obtain the activated-quenched polysaccharide.

In an embodiment, the polysaccharides are subjected to an “activation” step. The term “activation” refers to a chemical treatment of the polysaccharide to provide chemical groups capable of reacting with quenching agents. The native polysaccharides can also be subjected to preparation processes, such as sizing, before subjecting it to activation. The polysaccharide obtained upon activation is referred to as “activated polysaccharide”.

Polysaccharides can be activated using any of the reactions which are routinely used to activate the components for preparing a polysaccharide-protein conjugate vaccine composition. The most commonly employed methods for preparing polysaccharide protein conjugates include:
1) reductive amination chemistry, wherein the polysaccharide is activated by oxidation of hydroxyl groups, using oxidising agents such as TEMPO and periodates (metaperiodate, orthoperiodate, periodic acid sodium periodate or potassium periodate etc.), to form reactive aldehyde group and the aldehyde group of activated polysaccharide reacts with the amino or hydrazide group on the other component (amino acid or protein), and the C═N double bond formed is subsequently reduced to C—N single bond by a reducing agent such as cyanoborohydrides (such as sodium cyanoborohydride) or sodium borohydride;
2) cyanylation chemistry, wherein the polysaccharide is activated either by cyanogen bromide (CNBr) or by 1-cyano-4-dimethylammoniumpyridinium tetrafluoroborate (CDAP) to introduce a cyanate group on the hydroxyl group, which forms a covalent bond to the amino or hydrazide group upon addition of the protein component; and
3) a carbodiimide reaction, wherein carbodiimide activates the carboxyl group on one component of the conjugation reaction, and the activated carbonyl group reacts with the amino or hydrazide group on the other component. These reactions are also frequently employed to activate the components of the conjugate prior to the conjugation reaction. Briefly, condensing reagents such as 1-Ethyl-3-(3-dimethylaminopropyl)carbodiimide (EDC) or dicyclohexyl carbodiimide (DCC) activates the carboxyl group of the polysaccharide, which is followed by quenching with amino acid or protein having primary amines.

In a yet another embodiment, the present invention provides a method for preparing the reference standard, comprising of an activated-quenched polysaccharide, wherein the said method comprises the steps of:
a) treating a polysaccharide with 1-cyano-4-dimethylamino-pyridinium tetrafluoroborate (CDAP) to obtain an activated polysaccharide; and
b) quenching the activated polysaccharide from step (a) with an amino acid to obtain the activated-quenched polysaccharide;
   wherein, the reference standard is used for quantifying polysaccharides in a conjugate vaccine formulation, using nephelometry.

The activation of polysaccharides is carried out using the methods known in the art.

The activated polysaccharide is subsequently quenched to obtain the activated-quenched polysaccharide. The term “quenching” refers to the inactivation of the activated groups on the activated polysaccharide. The activated polysaccharide, used to prepare the reference standard of the present invention, is not reacted with any carrier protein before quenching with the quenching agent.

The activated polysaccharide is quenched by adding a quenching agent, which may be an amino acid selected from glycine, lysine, alanine and the like to the activation reaction mixture.

In a preferred embodiment, the activated polysaccharide is quenched by adding glycine to the activated polysaccharide.

In an embodiment, the concentration of glycine solution added to the activation reaction mixture is between 2 μM to 5.5 μM.

In an embodiment, quenching is carried out at a pH ranging from about 8.5 to about 9.5. The pH is adjusted using triethylamine or glacial acetic acid.

In an embodiment, the ratio of activated polysaccharide to glycine ranges from 0.6 to 1.5.

In an embodiment, the quenching is carried out for a time period ranging from 2 hour to 16 hours.

After quenching the activated polysaccharide, the resultant activated-quenched polysaccharide may be purified using conventional techniques to remove the residual reagents from the reaction mixture. These techniques include concentration/diafiltration, precipitation/elution, column chromatography, depth filtration and the like.

In an embodiment, the activated-quenched polysaccharide, is diluted with a buffer selected from succinate, phosphate and the like.

The concentration of buffer used may range from 5 mM to 7 mM. The pH of the buffer used for diluting the polysaccharide ranges from 5.5 to 6.5.

In an embodiment, the diluted activated-quenched polysaccharide is subsequently concentrated using Molecular Weight Cut-Off (MWCO) filter and then diafiltered using a buffer to remove the residual reagents/chemicals.

In an embodiment, a MWCO filter of size ranging from 10 KDa to 100 kDa is used.

Suitable buffers for diafilteration include, but are not limited to, succinate, Na₂CO₃, 3-(cyclohexylamino)-1-propanesulfonicacid (CAPS), and (2-(N-cyclohexylamino)ethane sulfonic acid (CHES) and the like.

The activated-quenched polysaccharide obtained after diafiltration can be further purified using a membrane. In a preferred embodiment, the diafiltered activated-quenched polysaccharide is further purified using a 0.2n, filter.

In an embodiment, the present invention provides a method for preparing the reference standard, comprising of an activated-quenched polysaccharide, wherein the said method comprises the steps of:
a) treating a polysaccharide with an activating agent selected from 1-cyano-4-dimethylamino-pyridinium tetrafluoroborate (CDAP), to obtain an activated polysaccharide; and
b) quenching the activated polysaccharide from step (a) with glycine to obtain the activated-quenched polysaccharide.

In an embodiment, the present invention provides a method for preparing the reference standard, comprising of an activated-quenched polysaccharide, wherein the said method comprises the steps of:
a) treating a polysaccharide with an activating agent selected from 1-cyano-4-dimethylamino-pyridinium tetrafluoroborate (CDAP) to obtain an activated polysaccharide, wherein the polysaccharide comprises of pneumococcal capsular polysaccharide of one or more serotypes selected from 1, 3, 4, 5, 6B, 7F, 9V, 14, 18C, 19A, 19F, 22F, 23F and 33F; and
b) quenching the activated polysaccharide from step (a) with glycine to obtain the activated-quenched polysaccharide.

In an embodiment, the present invention provides a method for preparing the reference standard, comprising of an activated-quenched polysaccharide, wherein the said method comprises the steps of:
a) treating a polysaccharide with 1-cyano-4-dimethylamino-pyridinium tetrafluoroborate (CDAP) and
b) quenching the activated polysaccharide from step (a) with glycine at a pH ranging from 8.5 to 9.5 and a temperature range of 22 to 28° C. for a time period ranging from 2 to 16 hours, to obtain the activated-quenched polysaccharide.

In an embodiment, the present invention provides a method for preparing the reference standard, comprising of an activated-quenched polysaccharide, wherein the said method comprises the steps of:
a) treating a pneumococcal capsular polysaccharide of serotypes selected from 1, 3, 4, 5, 6B, 7F, 9V, 14, 18C, 19A, 19F, 22F, 23F and 33F with 1-cyano-4-dimethylamino-pyridinium tetrafluoroborate (CDAP) to obtain an activated polysaccharide, and
b) quenching the activated polysaccharide from step (a) with glycine at a pH ranging from 8.5 to 9.5 and a temperature range of 22 to 28° C. for a time period ranging from 2 to 16 hours, to obtain the activated-quenched polysaccharide.

In an embodiment, the reference standard of the present invention, comprising of activated-quenched polysaccharide, remains stable for a time period of at least 3 months.

In an embodiment, the present invention also provides a nephelometry based method for quantifying polysaccharides in a conjugate vaccine composition using the reference standard comprising of the activated-quenched polysaccharide.

The reference standard used for quantification have to be assigned an unitage using methods selected from Thermal Gravimetric analysis, anthrone based method or 2-Phenoxyethanol (2-PE) based method for quantifying the polysaccharide content.

The native polysaccharide reference standard has been assigned a unitage by Thermal Gravimetric Analysis and anthrone method. The polysaccharide-protein conjugate reference standard has been assigned unitage using anthrone based method for quantifying polysaccharide (Rajendar B et al., Analytical Biochemistry, 2020 April; 595).

In a preferred embodiment, the activated-quenched polysaccharide based reference standard of the invention have been assigned unitage using 2-PE based method for quantification of polysaccharide. ((Burki et al., Analytical Biochemistry. 2020 April; 595.)

The reference standard of the present invention is used to develop standard curves for a specific concentration range, to estimate the concentration of polysaccharide in a conjugate vaccine drug product, using nephelometry.

To prepare the standard curves, the activated quenched polysaccharide standard was prepared from the concentration mentioned on the vial of each of the 14 serotypes, wherein the vial concentration of the activated quenched polysaccharide standard, for each serotype, is determined by 2-PE method. Initially the stock reference standard of 1 mg/mL was prepared by diluting the bulk of each of the serotype 1, 3, 4, 5, 6B, 7F, 9V, 14, 18C, 19A, 19F, 22F, 23F and 33F using 1×PBS pH 7.2. The 1 mg/mL stock standard is further diluted to the working standard range of 0, 1, 2, 3.5, 5, 7 and 10 μg/mL using 1×PBS pH 7.2, and a standard curve is prepared for the range.

In one embodiment, the invention provides a method for quantification of polysaccharides in a drug product using an activated-quenched polysaccharide of the present invention. In an embodiment, the invention provides a method for quantifying the polysaccharide content in a vaccine drug product, the said method comprising the steps of:
a. reacting the polysaccharide specific sera with various pre-determined concentrations of the reference standard of the present invention, and determining the light-scattering rate in a nephelometer to obtain a standard curve;
b. reacting the conjugate vaccine drug product with polysaccahride specific sera and determining the light-scattering rate in a nephelometer; and
c. comparing the light-scattering rate obtained from step b. with the standard curve obtained from step a. to obtain the polysaccharide content in the conjugate vaccine composition.

In an embodiment, the activated-quenched polysaccahrides of the present invention can be used as a reference standard for quantification of polysaccharide in conjugate as was as unconjugated vaccine composition as per the above mentioned process.

In an embodiment, the reference standard of the present invention, comprising of the activated-quenched polysaccharide, is used to quantify the polysaccharide content in a conjugate vaccine drug product, wherein the polysaccharide in the conjugate vaccine drug product is from Neisseria meningitidis, Streptococcus pneumoniae, Haemophilus influenzae type b, or Salmonella typhi.

In a preferred embodiment, the reference standard of the present invention, comprising of the activated-quenched polysaccharides, is used for the determination of polysaccharide content from individual serotype in a multivalent Pneumococcal Conjugate Vaccine (PCV) drug product.

In yet another embodiment, the multivalent Pneumococcal conjugate vaccine adsorbed onto aluminium phosphate is subjected to individual serotype saccharide content analysis using activated-quenched polysaccharide as a reference standard.

In an embodiment, the reference standard of the present invention, comprising of activated-quenched polysaccharides, is used for determination of individual polysaccharide content of each serotype in a multivalent Pneumococcal Conjugate Vaccine (PCV) drug product, wherein the multivalent (PCV) drug product is a 10 valent, 11 valent, 12 valent, 13 valent, 14 valent, 15 valent, 20 valent, 22 vlaent, 24 valent, 25 valent, 26 valent, 28 valent, 30 valent, 32 valent or 34 valent composition, comprising capsular polysaccharide from 1, 2, 3, 4, 5, 6A, 6B, 6C, 7F, 8, 9N, 9V, 10A, 11A, 12F, 14, 15A, 15B, 15C, 16F, 17F, 18C, 19F, 19A, 20A, 20B, 22F, 23A, 23B, 23F, 24B, 24F, 31, 33F, 34, 35B, 35F, 38, 39 and 45 and the like.

In an embodiment, the polysaccahrides are capsular polysaccahrides from one or more pneumococcal serotypes selected from 1, 2, 3, 4, 5, 6A, 6B, 6C, 6D, 6E, 6G, 6H, 7A, 7B, 7C, 7F, 8, 9A, 9L, 9F, 9N, 9V, 10A, 10B, 10C, 10D, 10F, 11A, 11F, 11B, 11C, 11D, 11E, 12A, 12B, 12F, 13, 14, 15A, 15C, 15B, 15F, 16A, 16F, 17A, 17F, 18C, 18F, 18A, 18B, 19A, 19B, 19C, 19F, 20, 20A, 20B, 21, 22A, 22F, 23A, 23B, 23F, 24A, 24B, 24F, 25F, 25A, 27, 28F, 28A, 29, 31, 32A, 32F, 33A, 33C, 33D, 33E, 33F, 33B, 34, 45, 38, 35A, 35B, 35C, 35F, 36, 37, 38, 39, 40, 41F, 41A, 42, 43, 44, 45, 46, 47F, 47A, and 48

In an embodiment, the polysaccharides of the multivalent PCV drug product are conjugated to one or more carrier protein selected from CRM197, PsaA, or PspA.

In an embodiment, the activated-quenched polysaccharides of the present invention are used as a reference standard for determination of individual polysaccharide serotype content in a multivalent Pneumococcal Conjugate Vaccine (PCV) drug product, comprising of capsular polysaccharide from pneumococcal serotypes 1, 3, 4, 5, 6B, 7F, 9V, 14, 18C, 19A, 19F, 22F, 23F and 33F.

In an exemplary embodiment, the activated-quenched polysaccharides of the present invention are used as a reference standard for determination of individual serotype content in a multivalent Pneumococcal Conjugate Vaccine (PCV) drug product, comprising of capsular polysaccharide serotypes 1, 3, 4, 5, 6B, 7F, 9V, 14, 18C, 19A, 19F, 22F, 23F and 33F individually conjugated to carrier protein CRM₁₉₇.

In an embodiment, the activated-quenched polysaccharides of the present invention are used as a reference standard for determination of individual polysaccharide serotype content in a multivalent meningococcal vaccine composition comprising one or more serotypes from A, C, W, X, Y and the like.

In an embodiment, the activated-quenched polysaccharides of the present invention are used as a reference standard for determination of individual polysaccharide serotype content in a monovalent or bivalent vaccine composition comprising polysaccharide selected from Vi and/or 02.

In one embodiment, the invention provides a method for the preparation of the sera for the quantification of the individual serotype polysaccharide content in the drug product. The sera was subjected to 1:5 dilution using 10 mM PBS containing 1% PEG and 0.2% Tween-20 and preadsorbed by the addition of 10 μg/mL of 13 valent polysaccharide mix except the target serotype polysaccharide. The sera mix was incubated for 30 min on a rocker and clarified at 16000 g for 30 min. The clarified sera was used for the analysis.

In the present embodiment, the conjugate vaccine drug product was subjected to trypsin and/or alkali treatment, prior to quantifying the polysaccharide content in the drug product. For the trypsin treatment, the conjugate vaccine drug product was diluted with 10 mM Tris buffer pH 9 followed by treatment with 0.1 mg/mL trypsin overnight at 37° C. under 200 rpm/min. The treated sample was centrifuged at 16000 g for 30 min and the clarified supernatant was used for evaluation. In alkali treatment, the conjugate vaccine drug product was added to 1M NaOH solution, incubated for 30 sec and immediately neutralised with 1M Citric acid and the pH was adjusted to 9 with 1M citric acid. The treated sample was evaluated using polyvalent standard in the range of 0-10 μg/mL.

In the present embodiment, a nephelometry method was developed for the determination of the individual serotype polysaccharide content in the conjugate vaccine. In the nephelometry based method of the present invention, for quantifying the polysaccharide in the conjugate vaccine drug product, 14 μL of the reference standards, samples and controls were added in the respective wells as per the plate layout followed by addition of 35 μL of 1:5 diluted sera and 179 μL of 1×PBS pH 7.2 to all the wells. The plate was gently tapped on the sides and incubated for 30 min on plate shaker at 200 rpm followed by reading in the microplate Nephelometer to attain a stable reading and the data was compiled.

The following examples are provided to illustrate the invention and are merely for illustrative purpose only and should not be constructed to limit the scope of the invention.

### EXAMPLES

### Example 1: Quantification of Polysaccharides in a Drug Product by Nephelometry Using Native Polysaccharides as Reference Standard

To meet the compendia (IP/BP/WHO TRS 977) requirement Nephelometry assay using native polysaccharide as standard with an assigned unitage by Thermal Gravimetric Analysis, 2-PE, Anthrone method was carried out. (Rajendar B et al., Analytical Biochemistry, 2020 April; 595). The individual serotype specific saccharide content in the multivalent PCV drug product, comprising of serotypes 1, 3, 4, 5, 6B, 7F, 9V, 14, 18C, 19A, 19F, 22F, 23F and 33F, each serotype conjugated to CRM₁₉₇ carrier protein, was determined using nephelometry. The conjugated vaccine was formulated with serotypes 1, 3, 4, 5, 7F, 9V, 14, 18C, 19A, 19F, 22F, 23F and 33F at 4.4 μg/mL whereas 6B at 8.8 μg/mL.

Each of the serotype polysaccharide specific sera was subjected to 1:5 dilution using 10 mM PBS containing 1% PEG and 0.2% Tween-20 and preadsorbed by the addition of 10 μg/mL of 13 valent polysaccharide mix except the target serotype polysaccharide. The sera mix was incubated for 30 min on a rocker and clarified at 16000 g for 30 min. The clarified sera was used for the nepheloemtry analysis.

Standard curves were prepared for serotypes 1, 3, 4, 5, 6B, 7F, 9V, 14, 18C, 19A, 19F, 22F, 23F and 33F using the native polysaccharide as the reference standard at polysaccharide concentrations of 0, 1, 2, 3.5, 5, 7 and 10 μg/mL. 14 μL of the reference standards, and controls were added in the respective wells as per the plate layout followed by addition of 35 μL of 1:5 diluted sera and 179 μL of 1×PBS pH 7.2 to all the wells. The plate was gently tapped on the sides and incubated for 30 min on plate shaker at 200 rpm followed by reading in the light scattering rate in nephelometer.

The multivalent PCV drug product was desorbed using trypsin. The multivalent PCV drug product was diluted with 10 mM Tris buffer pH 9 followed by treatment with 0.1 mg/mL trypsin overnight at 37° C. under 200 rpm/min.

14 μL of the desorbed conjugate vaccine drug product and controls were added in the respective wells as per the plate layout followed by addition of 35 μL of 1:5 diluted sera and

179 μL of 1×PBS pH 7.2 to all the wells. The plate was gently tapped on the sides and incubated for 30 min on plate shaker at 200 rpm followed by reading the light scattering rate in nephelometer, to obtain the concentration of polysaccharide in the multivalent PCV drug product. The individual serotype specific saccharide content in the drug product was determined against the reference standard curve in the range of 0-10 μg/mL.

Result:

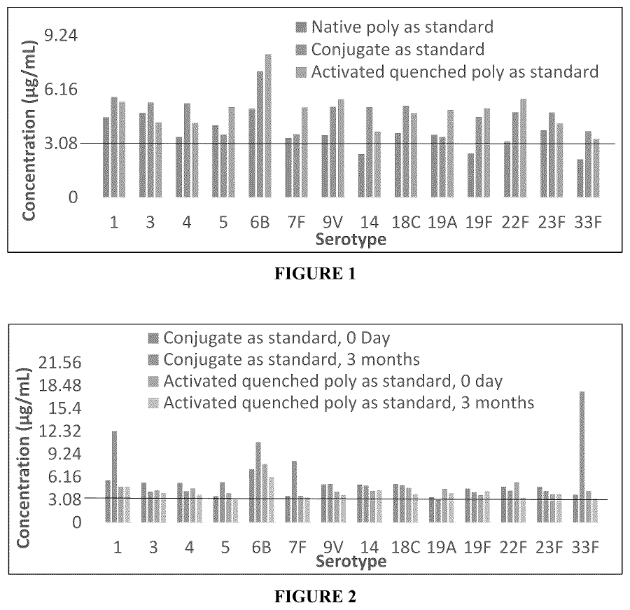

The individual serotype saccharide content in the drug product using native polysaccharide as reference material is illustrated in FIG. 1.

The saccharide content of serotypes 14, 19F and 33F were not meeting the acceptance criteria of 70-130% of the label claim. Since the batch formulation is being done using conjugate material (Polysaccharide conjugation with CRM₁₉₇) may be native polysaccharide is not the best choice for usage as standard by Nephelometry.

### Example 2: Quantification of Polysaccharides in a Drug Product by Nephelometry Using Polysaccharide-Protein Conjugate as Reference Standard

The polysaccharide-protein conjugate reference standard, comprising of pneumococcal serotype polysaccharides conjugated to CRM₁₉₇, were assigned unitage using anthrone based method for quantifying polysaccharides. The individual serotype specific saccharide content in the multivalent PCV drug product, comprising of serotypes 1, 3, 4, 5, 6B, 7F, 9V, 14, 18C, 19A, 19F, 22F, 23F and 33F, each serotype conjugated to CRM₁₉₇ carrier protein, was determined using nephelometry. The conjugated vaccine was formulated with serotypes 1, 3, 4, 5, 7F, 9V, 14, 18C, 19A, 19F, 22F, 23F and 33F at 4.4 μg/mL whereas 6B at 8.8 μg/mL. Each of the serotype polysaccharide specific sera was subjected to 1:5 dilution using 10 mM PBS containing 1% PEG and 0.2% Tween-20 and preadsorbed by the addition of 10 μg/mL of 13 valent polysaccharide mix except the target serotype polysaccharide. The sera mix was incubated for 30 min on a rocker and clarified at 16000 g for 30 min. The clarified sera was used for the nepheloemtry analysis.

Standard curves were prepared for serotype polysaccharide 1, 3, 4, 5, 6B, 7F, 9V, 14, 18C, 19A, 19F, 22F, 23F and 33F using the individual polysaccharide-protein glycoconjugate as the reference standard at concentrations of 0, 1, 2, 3.5, 5, 7 and 10 μg/mL. 14 μL of the reference standards, and controls were added in the respective wells as per the plate layout followed by addition of 35 μL of 1:5 diluted sera and 179 μL of 1×PBS pH 7.2 to all the wells. The plate was gently tapped on the sides and incubated for 30 min on plate shaker at 200 rpm followed by reading in the light scattering rate in nephelometer.

The multivalent PCV drug product was desorbed using trypsin. The multivalent PCV drug product was diluted with 10 mM Tris buffer pH 9 followed by treatment with 0.1 mg/mL trypsin overnight at 37° C. under 200 rpm/min. 14 μL of the desorbed conjugate vaccine drug product and controls were added in the respective wells as per the plate layout followed by addition of 35 μL of 1:5 diluted sera and 179 μL of 1×PBS pH 7.2 to all the wells. The plate was gently tapped on the sides and incubated for 30 min on plate shaker at 200 rpm followed by reading the light scattering rate in nephelometer, to obtain the concentration of polysaccharide in the multivalent PCV drug product. The individual serotype specific saccharide content in the drug product was determined against the reference standard curve.

Result:

The data using glycoconjugate as reference standard is illustrated in FIG. 1. The saccharide content of all the serotypes in the drug product were within the acceptance criteria of 70-130% of the label claim, when glycoconjugate is used as reference standard.

### Example 3: Generation of Activated-Quenched Polysaccharide for Use as Reference Standard

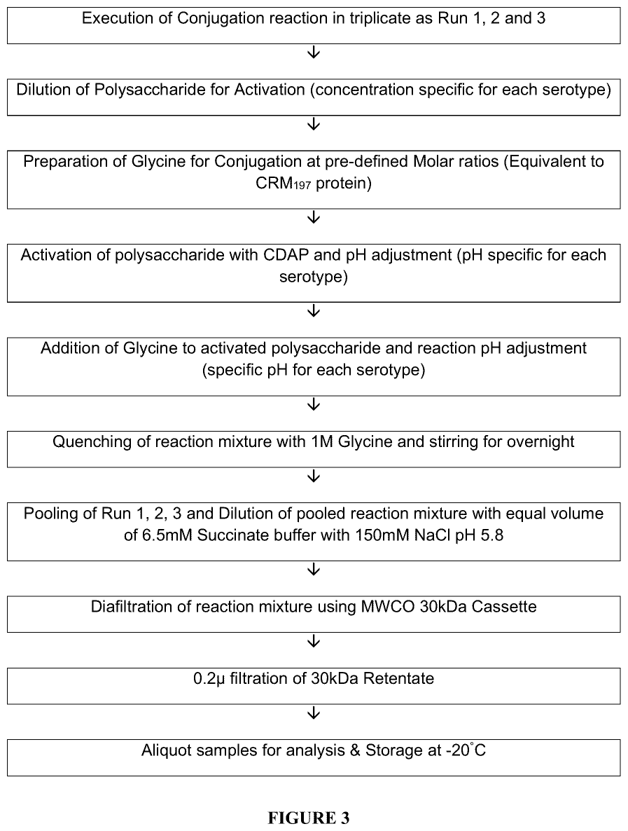

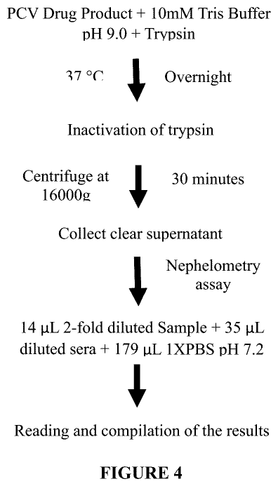

Pneumococcal polysaccharides of serotype 1, 3, 4, 5, 6B, 7F, 9V, 14, 18C, 19A, 19F, 22F, 23F and 33F were subjected to activation using CDAP and quenched by addition of Glycine as mentioned in the process (Refer FIG. 3). Activation of the polysaccharide was performed at 200 mg scale in triplicate as run 1, 2 and 3 (After reaction, all the 3 runs were pooled and diafiltered). The polysaccharide bulk was thawed at RT (22±3° C.) and diluted using WFI and 5M NaCl to the required activation concentration-specific for each serotype. Activation was performed using CDAP (100 mg/mL w/v in Acetonitrile) in pre-defined ratio specific for each serotype (1:0.2-1.0) and the pH (specific for each polysaccharide) 8.5 to 9.5 was adjusted using 0.2M Triethylamine. Glycine stock (10 mg/mL) was diluted to the molar ratios equivalent to CRM₁₉₇ protein using Water For Injection (WFI) and 5M NaCl to required volume specific for each serotype and added to the reaction mixture (The quantity of glycine is being used in the reaction is equivalent to the amount of protein which is used in each serotype conjugation reaction. The glycine concentration ranges from 2-5.5 μM. pH of the reaction mixture (specific for each serotype) was adjusted in the range of 8.5 to 9.5 using 0.2M Triethylamine/1M Glacial Acetic Acid and incubated at RT for 5 Hrs. Reaction was quenched by adding 1M Glycine (Volume equal to 1/10^{th} of the reaction volume).

Run 1, 2 and 3 were pooled and diluted with equal volume of 6.5 mM Succinate buffer containing 150 mM NaCl at pH 5.8. Pooled activation reaction mixture was diafiltered using Molecular Weight Cut-Off (MWCO) 30 kDa Tangential Flow Filtration (TFF) cassette and diafiltered using 6.5 mM succinate buffer containing 150 mM NaCl at pH 5.8 to remove residual reagents/chemicals (TEA, CDAP, ACN etc.). Thereafter, the retentate was collected into a presterilized glass bottle.

Volume of retentate was made up to 270-290 mL approximately with 6.5 mM succinate buffer at pH 5.8 containing 150 mM NaCl and filtered through 0.211 filter (Millex GP Syringe Filter (33 mm), MOC: PES Membrane) into a sterile PETG bottle.

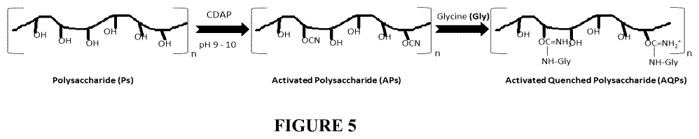

FIG. 5 depicts the schematic illustration of preparation of activated quenched polysaccharide and the activated-quenched polysaccharide obtained from the process disclosed in this example.

### Example 4: Quantification of Polysaccharides in a Drug Product by Nephelometry Using Activated-Quenched Polysaccharide as Reference Standard

The unitage of activated quenched polysaccharide reference material was assigned by 2PE method and used for the determination of the individual serotype saccharide content in the multivalent PCV drug product (Rajendar B et al., Analytical Biochemistry, 2020 April; 595). The individual serotype specific saccharide content in the multivalent PCV drug product, comprising of serotypes 1, 3, 4, 5, 6B, 7F, 9V, 14, 18C, 19A, 19F, 22F, 23F and 33F, each serotype conjugated to CRM₁₉₇ carrier protein, was determined using nephelometry. The conjugated vaccine was formulated with serotypes 1, 3, 4, 5, 7F, 9V, 14, 18C, 19A, 19F, 22F, 23F and 33F at 4.4 μg/mL whereas 6B at 8.8 μg/mL.

Each of the serotype polysaccharide specific sera was subjected to 1:5 dilution using 10 mM PBS containing 1% PEG and 0.2% Tween-20 and preadsorbed by the addition of 10 μg/mL of 13 valent polysaccharide mix except the target serotype polysaccharide. The sera mix was incubated for 30 min on a rocker and clarified at 16000 g for 30 min. The clarified sera was used for the analysis.

Standard curves were prepared for serotype polysaccharide 1, 3, 4, 5, 6B, 7F, 9V, 14, 18C, 19A, 19F, 22F, 23F and 33F using the individual activated-quenched polysaccharide as the reference standard at concentrations of 0, 1, 2, 3.5, 5, 7 and 10 μg/mL. 14 μL of the reference standards, and controls were added in the respective wells as per the plate layout followed by addition of 35 μL of 1:5 diluted sera and 179 μL of 1×PBS pH 7.2 to all the wells. The plate was gently tapped on the sides and incubated for 30 min on plate shaker at 200 rpm followed by reading in the light scattering rate in nephelometer.

The multivalent PCV drug product was desorbed using trypsin. The multivalent PCV drug product was diluted with 10 mM Tris buffer pH 9 followed by treatment with 0.1 mg/mL trypsin overnight at 37° C. under 200 rpm/min.

14 μL of the desorbed conjugate vaccine drug product and controls were added in the respective wells as per the plate layout followed by addition of 35 μL of 1:5 diluted sera and 179 μL of 1×PBS pH 7.2 to all the wells. The plate was gently tapped on the sides and incubated for 30 min on plate shaker at 200 rpm followed by reading the light scattering rate in nephelometer, to obtain the concentration of polysaccharide in the multivalent PCV drug product. The individual serotype-specific saccharide content in the drug product was determined against the reference standard curve.

Result:

The data of individual serotype saccharide content in the drug product using activated quenched polysaccharide as reference standard material with 2-PE input is illustrated in FIG. 1. The saccharide content of all the serotypes in the drug product were found to be within the acceptance criteria of 70-130% of the label claim, when activated-quenched polysaccharide was used as reference standard.

### Example 5: Stability of Reference Standard (Conjugate and Activated Quenched Polysaccharide)

Stability of the conjugate and activated quenched polysaccharide reference standard was performed at 0 and 3 months' time point and used for determination of the individual serotype saccharide content in the drug product.

The data of the individual serotype saccharide content in the drug product using 0 day and 3 months old conjugate and activated quenched polysaccharide reference standard material is illustrated in FIG. 2.

Higher saccharide contents of serotypes 1, 7F and 33F were observed using 3 month conjugate reference standard indicating the aggregation or degradation of the standard whereas saccharide content of all the serotypes were meeting the specification using activated quenched polysaccharide as standard till 3rd month and the results are comparable with the 0 day results.

Evaluation of the native polysaccharide, conjugate material and activated quenched polysaccharide as reference standard for the analysis of the drug product shows that, activated quenched polysaccharide with the input by 2-PE method compared to all other standards resulted in individual saccharide content meeting the specification of 70-130% of the label claim. Hence, activated quenched polysaccharide can be used as the official test and reference standard for information gathering for all release and stability time points of the drug product.

### Advantage of the Invention

The present invention provides improved activated-quenched polysaccharide which are stable and can be used for accurate quantification of polysaccharides in vaccine compositions. The invention also provides a simple, cost-effective and labour efficient method for preparing the activated-quenched polysaccharide. The invention further provides improved methods for quantification of polysaccharide in a conjugate vaccine composition using the activated-quenched polysaccharide of the present invention in a nephelometry process.

1. A reference standard for quantifying polysaccharide in a vaccine composition, wherein the said reference standard comprises of an activated-quenched polysaccharide.

2. The reference standard of claim 1, wherein an activated polysaccharide is quenched using a quenching agent.

3. The reference standard of claim 2, wherein the quenching agent is an amino acid selected from a group comprising glycine, lysine, alanine and the like.

4. The reference standard of claim 3, wherein the ratio of activated polysaccharide to amino acid ranges from 0.6 to 1.5.

5. The reference standard of claim 1, wherein the polysaccharide of the activated-quenched polysaccharide is from a bacterial source selected from a group comprising of Neisseria meningitidis, Streptococcus pneumoniae, Haemophilus influenzae type b, and Salmonella typhi.

6. The reference standard of claim 5, wherein the polysaccharide is capsular polysaccharide from one or more serotypes of Streptococcus pneumoniae, wherein the serotypes are selected from the group comprising of 1, 2, 3, 4, 5, 6A, 6B, 6C, 7F, 8, 9N, 9V, 10A, 11A, 12F, 14, 15A, 15B, 15C, 16F, 17F, 18C, 19F, 19A, 20A, 20B, 22F, 23A, 23B, 23F, 24B, 24F, 31, 33F, 34, 35B, 35F, 38, 39 and 45 and the like.

7. The reference standard of claim 5, wherein the polysaccharide is from one or more serotypes of Neisseria meningitides, wherein the serotypes are selected from A, C, W, X, Y and the like.

8. The reference standard of any one of the claims 1-6, wherein the said reference standard remains stable for a time period of at least 3 months.

9. A method for preparing a reference standard, comprising of an activated-quenched polysaccharide, for quantifying polysaccharide in a vaccine composition, wherein the said method comprises the steps of:
a. treating a polysaccharide with an activating agent to obtain an activated polysaccharide; and
b. quenching the activated polysaccharide from step (a) with a quenching agent to obtain the activated-quenched polysaccharide.

10. The method of claim 9, wherein the activating agent is selected from a cyanylating agent, oxidising agent, reducing agent and condensing reagents.

11. The method of claim 10, wherein the cyanylating agent is selected from group comprising 1-cyano-4-dimethylamino-pyridinium tetrafluoroborate (CDAP), cyanogen bromide, N-cyano trimethyl ammonium tetrafluoroborate (CTEA) and p-nitro phenyl cyanate (pNPC), preferably CDAP.

12. The method of claim 9, wherein the ratio of the polysaccharide to activating agent in step a) ranges from 1:0.2-1.0.

13. The method of claim 9, wherein the quenching agent is an amino acid selected from a group comprising glycine, lysine, alanine and the like.

14. The method of claim 9, wherein the ratio of activated polysaccharide to amino acid ranges from 0.6 to 1.5.

15. The method of claim 9, wherein the quenching is carried out at a pH ranging from about 8.5 to about 9.5, temperature ranging from 22 to 28° C. and for a time period ranging from 2 to 16 hours.

16. The method of claim 9, wherein the polysaccharide is from bacterial source selected from a group comprising of Neisseria meningitidis, Streptococcus pneumoniae, Haemophilus influenzae type b, and Salmonella typhi.

17. The method of claim 16, wherein the polysaccharide is capsular polysaccharide from one or more serotypes of Streptococcus pneumoniae, wherein the serotypes are selected from group comprising 1, 2, 3, 4, 5, 6A, 6B, 6C, 7F, 8, 9N, 9V, 10A, 11A, 12F, 14, 15A, 15B, 15C, 16F, 17F, 18C, 19F, 19A, 20A, 20B, 22F, 23A, 23B, 23F, 24B, 24F, 31, 33F, 34, 35B, 35F, 38, 39, 45 and the like.

18. The method of claim 16, wherein the polysaccharide is from one or more serotypes of Neisseria meningitides, wherein the serotypes are selected from A, C, W, X, Y and the like.

19. A method for quantifying the polysaccharide content in a vaccine drug product, the said method comprising the steps of:
a. reacting the polysaccharide specific sera with various pre-determined concentrations of specific reference standard of any one of claims 1-7, and determining the light-scattering rate in a nephelometer to obtain a standard curve;
b. reacting the vaccine drug product with polysaccharide specific sera and determining the light-scattering rate in a nephelometer; and
c. comparing the light-scattering rate obtained from step b. with the standard curve obtained from step a. to obtain the polysaccharide content in the vaccine composition.

20. The method of claim 19, wherein the vaccine drug product is subjected to desorption treatment prior to reacting with polysaccharide specific sera.

21. The method of claim 20, wherein the vaccine drug product is desorbed by trypsin treatment.

22. The method of claim 19, wherein the polysaccharide in the vaccine drug product is from Neisseria meningitidis, Streptococcus pneumoniae, Haemophilus influenzae type b, or Salmonella typhi.

23. The method of claim 19, wherein the vaccine drug product is a multivalent pneumococcal conjugate vaccine drug product.

24. The method of claim 23, wherein the multivalent (PCV) drug product is a 10 valent, 11 valent, 12 valent, 13 valent, 14 valent, 15 valent, 20 valent, 22 vlaent, 24 valent, 25 valent, 26 valent, 28 valent, 30 valent, 32 valent, or 34 valent composition.

25. The method of claim 24, wherein the multivalent pneumococcal conjugate vaccine drug product comprises of polysaccharides from one or more pneumococcal serotypes selected from 1, 2, 3, 4, 5, 6A, 6B, 6C, 7F, 8, 9N, 9V, 10A, 11A, 12F, 14, 15A, 15B, 15C, 16F, 17F, 18C, 19F, 19A, 20A, 20B, 22F, 23A, 23B, 23F, 24B, 24F, 31, 33F, 34, 35B, 35F, 38, 39, 45 and the like.

26. The method of claim 25, wherein the multivalent pneumococcal conjugate vaccine drug product comprises of capsular polysaccharides from pneumococcal serotypes 1, 3, 4, 5, 6B, 7F, 9V, 14, 18C, 19A, 19F, 22F, 23F and 33F.

27. The method of claim 25, wherein the polysaccharides of the multivalent pneumococcal conjugate vaccine drug product are conjugated to one or more carrier proteins selected from CRM197, PsaA, or PspA, preferably CRM197.

28. The method of claim 22, polysaccharide is from from Neisseria meningitides.
